(19) United States
(12) Patent Application Publication
(10) Pub. No.: US 2001/0021503 A1
(43) Pub. Date: Sep. 13, 2001

(54) Oligomeric carrier molecules with defined incorporated marker groups and haptens

(75) Inventors: Hans-Peter Josel, Weilheim (DE); Andreas Finke, Penzberg (DE); Rupert Herrmann, Weilheim (DE); Eva Hoss, Starnberg (DE); Andreas Marschall, Weinheim (DE); Christoph Seidel, Weilheim (DE)

Correspondence Address: Roche Diagnostics Corporation, 9115 Hague Road, Bldg. D, P.O. Box 50457, Indianapolis, IN, 46250-0457, US

(73) Assignee: Boehringer Mannheim GmbH (4pp)

(21) Appl. No.: 09/801,157

(22) Filed: Mar. 7, 2001

Related U.S. Application Data
(62) Division of application No. 08/776,190, filed on Jan. 24, 1997.
Application No. 08/776,190 is a 371 of international application No. PCT/EP95/02915, filed on Jul. 24, 1995.

(30) Foreign Application Priority Data
Jul. 25, 1994 (DE) P 4426276.0
Aug. 31, 1994 (DE) P 4430998.8
Aug. 31, 1994 (DE) P 4430973.2
Nov. 4, 1994 (DE) P 4439345.8

(51) Int. Cl. C12Q 1/68; C12P 21/04

(52) U.S. Cl. 435/6; 525/54.11; 525/54.2

(57) ABSTRACT

The present invention concerns new conjugates, processes for their production as well as the use of these conjugates as antigens in immunological detection methods or for DNA diagnostics.

The present invention concerns new conjugates, processes for their production as well as the use of these conjugates as antigens in immunological methods of detection or for DNA diagnostics.

The detection of immunoglobulins in body fluids, in particular in human sera, is used to diagnose infections with microorganisms, in particular viruses, such as HIV, hepatitis viruses etc. The presence of specific immunoglobulins in the examined sample is usually detected by reaction with one or several antigens that react with the specific immunoglobulins. Methods for the determination of specific immunoglobulins in the sample liquid must be sensitive, reliable, simple and rapid.

A further immunological method is a competitive immunoassay in which an analyte is detected qualitatively and quantitatively in such a way that a hapten that is immunologically analogous to the analyte and the analyte compete for binding sites on a receptor e.g. an antibody. The analyte analogue is in this case usually used in a labelled form or in a form capable of binding to a solid phase.

In recent years more and more detection systems based on non-radioactive marker groups have been developed in which the presence of an analyte, e.g. a specific antibody, in the examined sample can be determined with the aid of optical (e.g. luminescent or fluorescent), NMR-active or metal-precipitating detection systems.

EP-A-0 307 149 discloses an immunological test for an antibody in which two recombinant polypeptides are used as antigens one of which is immobilized on a solid phase and the other carries a marker group whereby both recombinant antigens are expressed in different organisms in order to increase the specificity of the test.

EP-A-0 366 673 discloses a method for the detection of antibodies in a sample in which an antibody is detected by reaction with a purified labelled antigen and with the same purified antigen in a solid phase-bound form. Human IgG is for example disclosed as an antigen.

EP-A-0 386 713 describes a method for the detection of antibodies against HIV using two solid supports in which various HIV antigens are immobilized on the two solid supports each of which is brought into contact with an aliquot of a sample and with a labelled HIV antigen wherein the presence of antibodies is detected by a positive reaction in at least one of the tests. Recombinantly produced polypeptides are disclosed as HIV antigens.

EP-A-0 507 586 describes a method for carrying out an immunological test for a specific immunoglobulin in which a sample is brought into contact with two antigens capable of binding the immunoglobulin, wherein the first antigen carries a group suitable for binding to a solid support and the second antigen carries a marker group. The marker group can be a direct marker group e.g. an enzyme, a chromogen, a metal particle, or also an indirect marker group i.e. the marker group attached to the antigen can react with a receptor for the marker group which in turn carries a signal-generating group. A fluorescein derivative is mentioned as an example of such an indirect marker group, the receptor of which is an antibody which in turn is coupled to an enzyme. Polypeptides such as the hepatitis B surface antigen are disclosed as antigens. SH groups are introduced into this antigen by derivatization which are used to couple the fluorescein.

EP-A-0 507 587 discloses a method which is specifically suitable for the detection of IgM antibodies in which the sample is incubated with a labelled antigen which is directed against the antibody to be detected and with a second antibody which is also directed against the antibody to be detected and is capable of binding to a solid phase.

EP-A-0 199 804 and EP-A-0 580 979 disclose an immunological method of detection using antigens which are labelled with luminescent metal chelate groups and in particular with ruthenium and osmium chelate groups. Immunoglobulins are used as antigens which are statistically labelled by reaction with activated metal complexes.

EP-A-0 178 450 discloses metal chelates in particular ruthenium complexes to which an immunologically active material for example an antibody can be coupled. Coupling is achieved by statistical reaction of the immunologically reactive material with the metal chelate.

EP-A-0 255 534 discloses a luminescence immunoassay using a metal chelate-coupled antigen or antibody. Coupling is for example achieved by statistical reaction of a metal chelate active ester derivative with an antibody.

WO 90/05301 discloses a method for the detection and for the quantitative determination of analytes by electrochemiluminescence using luminescent metal chelates which are coupled to (i) an added analyte, (ii) a binding partner of the analyte or (iii) a reactive component that can bind to (i) or (ii). Luminescence is measured after binding the metal chelates to activated and optionally magnetic microparticles.

In the immunological methods for detecting antibodies known from the state of the art polypeptide antigens are usually used which are normally produced by recombinant DNA methods. However, problems may occur when using such polypeptide antigens. Thus recombinant polypeptides can often only be produced in the form of fusion polypeptides in which case the fused part can lead to false positive results in the test. In addition polypeptides produced by recombinant expression often only have a very low stability in the sample solution and tend to aggregate. A further disadvantage is that it is often not possible to selectively and reproducibly introduce marker groups into such polypeptides.

Moreover the production of recombinant polypeptide antigens involves high costs and large variations in the immunological reactivity in different lots of the recombinant polypeptides can occur.

Even in competitive immunoassays which have very high requirements for sensitivity and precision it is often very difficult to achieve the required lower detection limits using known antigens when detecting analytes that are only present in very low concentrations such as estradiol or testosterone in particular with detection systems based on electrochemiluminescence.

The object of the present invention was therefore to provide a process with which antigens for immunological tests can be produced in a simple and efficient manner wherein the disadvantages of the antigens known from the state of the art are at least partially eliminated. In addition the process should enable a selective and reproducible introduction of marker groups into the antigens.

This problem is resolved by conjugates comprising a polymeric carrier with a maximum of 100 monomeric units which contains 1-10 hapten molecules and 1-10 marker or solid phase binding groups coupled to reactive side groups wherein the monomeric units are selected from amino acids, nucleotides and peptidic nucleic acids.

When using the conjugates according to the invention that contain 1-10 hapten molecules and a defined number of marker or solid phase binding groups as antigens in an immunological method of detection it is surprisingly possible to achieve a considerable higher sensitivity and precision and at the same time at a reduced lower detection limit compared to known monomeric and multimeric antigens. Moreover the conjugates according to the invention can be constructed in a simple manner by solid phase synthesis e.g. a peptide solid phase synthesis. For this monomeric units, e.g. amino acid derivatives, that are derivatized by a hapten molecule or a marker or solid phase binding group can be incorporated at predetermined positions. In addition it is possible to selectively incorporate additional haptens or marker or solid phase binding groups after completion of the solid phase synthesis at positions of the carrier chain at which monomers are located having free functional groups. This enables a defined and reproducible incorporation of hapten molecules and marker or solid phase binding groups into the conjugate. The distances between individual groups on the conjugate can be exactly defined and varied if necessary. The signal quenching can be kept low by selecting the distance of the marker groups on the conjugate so that the signal strength increases proportionaly to the number of marker groups. A defined spatial orientation of marker groups also contributes to the improvement of the signal strength e.g. in the case of helical carriers. The distances between marker groups are therefore preferably 3-6 or/and 13-16 monomeric units in the case of helical carriers e.g. single-stranded or double-stranded nucleic acids.

The polymeric carrier molecule which forms the backbone of the conjugate has a maximum length of 100 monomeric units preferably of 3-80 monomeric units and especially preferably of 5-60 monomeric units.

The monomeric units are selected from amino acids, nucleotides and peptidic nucleic acids. The polymeric carrier preferably comprises a peptide chain, preferably a linear peptide chain, composed of amino acids. However, the carrier can also be an oligonucleotide to the reactive side groups of which hapten molecules and marker or solid phase binding groups are coupled.

In addition the polymeric carrier can be composed of peptidic nucleic acids. Peptidic nucleic acids comprise a polyamide backbone made of the same or different monomeric units of the formula (CH₂)ₖ—CHR′—N[CO—(CH₂)ᵢ—L]—CH₂—(CH₂)ₘ—NH—CO—, in which L is selected from the group comprising hydrogen, phenyl, naturally-occurring nucleobases and non-naturally-occurring nucleobases, R′ is selected from the group comprising hydrogen and the side chains of amino acids, preferably α amino acids, that occur naturally or non-naturally, k and m are each independently 0 or 1 and i is independently 0 to 5. The hapten molecules and marker or solid phase binding groups can be coupled to the nucleobases or/and amino acid side chains of the peptidic nucleic acids. Peptidic nucleic acids and their production are described in WO92/20703. Reference is herewith made to this disclosure.

Even in the case of peptidic nucleic acids the carrier can be present as a single or double strand. Double-stranded carriers with at least one PNA strand e.g. a PNA strand and a nucleic acid strand e.g. a DNA strand are particularly preferred.

The conjugate contains 1-10 hapten molecules preferably 1-6 hapten molecules and especially preferably 1 or 2 hapten molecules. The hapten is preferably an immunological reactive molecule having a molecular mass of 100-2000 Da. Such haptens can for example be selected from pharmacological active substances such as antibiotics, opiates, amphetamines, barbiturates, cytostatic agents (e.g. gentamicin, tobramycin, vancomycin etc.) paracetamol, salicylates, phenytoin, quinine and quinine derivatives, theophyllin etc., hormones and metabolites such as sterols, bile acids, sexual hormones (e.g. estradiol, estriol, testosterone, progesterone, pregnenolone and derivatives thereof), corticoids (e.g. cortisol, corticosterone, cortisone and derivatives thereof), cardenolides and cardenolide-glycosides (e.g. digoxin, digoxigenin, strophanthin, bufadienolides etc.) steroid-sapogenines, steroid alkaloids, peptide hormones, creatinine, thyroid hormones (e.g. T₃, T₄), neurotransmitters (e.g. serotonin, choline, γ-aminobutyric acid), vitamins and mediators such as prostaglandins, leucotrienes, leucoendiines and thromboxanes.

On the other hand the hapten can also be selected from immunologically reactive peptide epitopes preferably having a length of up to 30 amino acids. Such peptide epitopes can for example be derived from pathogenic organisms e.g. bacteria, viruses and protozoa or from autoimmune antigens. The immunologically reactive peptide epitopes can for example be derived from viral antigens e.g. the amino acid sequences of HIV I, HIV II or hepatitis C virus (HCV).

In addition the hapten can also be selected from nucleic acids with a length of preferably up to 50 nucleotides that are complementary to a nucleic acid sequence which is to be detected in the sample. Finally the hapten can also be selected from peptidic nucleic acids with a length of up to 50 monomeric units.

Moreover the conjugate according to the invention contains 1-10 preferably 2-8 marker or solid phase binding groups. Preferred examples of marker groups are luminescent metal chelates and fluorescent labels. Preferred examples of solid phase binding groups are biotin and biotin analogues such as desthiobiotin and iminobiotin which can specifically react with streptavidin or avidin.

The hapten molecules and marker or solid phase binding groups are preferably coupled to the carrier chain via reactive amino or/and thiol side groups particularly preferably via reactive primary amino side groups. Such side groups can be produced by incorporating appropriate monomers e.g. amino acids such as lysine, ornithine, hydroxylysine or cysteine into the carrier chain.

In certain embodiments of the present invention it may be preferable to incorporate a spacer between the hapten and the marker or solid phase binding group and the carrier chain. The spacer is preferably flexible and has a chain length of preferably 3-30 atoms. The spacer particularly preferably contains hydrophilic groups such as oxyalkylene or/and hydroxy side groups.

Preferred marker groups are luminescent metal chelates i.e. metal chelates which can generate a detectable luminescence reaction. This luminescence reaction can for example be detected by fluorescence or by electrochemiluminescence measurement. The metal of these metal chelates is for example a transition metal or a rare earth metal. The metal is preferably ruthenium, osmium, rhenium, iridium, rhodium, platinum, indium, palladium, molybdenum, technetium, copper, chromium or tungsten. Ruthenium, iridium, rhenium, chromium and osmium are particularly preferred. Ruthenium is most preferred.

The ligands which form the metal chelate together with the metal are usually polydentate ligands i.e. ligands with several co-ordination sites. Polydentate ligands for example include aromatic and aliphatic ligands. Suitable aromatic polydentate ligands include aromatic heterocyclic ligands. Preferred aromatic heterocyclic ligands are polyheterocycles containing nitrogen such as for example bipyridyl, bipyrazyl, terpyridyl and phenanthrolyl. These ligands can for example contain substituents such as alkyl, substituted alkyl, aryl, substituted aryl, aralkyl, carboxylate, carboxyaldehyde, carboxamide, cyano, amino, hydroxy, imino, hydroxycarbonyl, aminocarbonyl, amidine, guanidinium, ureide, groups containing sulphur, groups containing phosphorus and the carboxylate ester of N-hydroxysuccinimide. Preferred ligands contain C₂-C₃ alkylenoxy, C₂-C₃ alkylenethio and C₂-C₃ alkylene amino groups, in particular ethylene-oxy groups. The chelate can also contain one or several monodentate ligands. Examples of monodentate ligands comprise carbon monoxide, cyanides, isocyanides, halogenides and aliphatic, aromatic and heterocyclic phosphines, amines, stilbenes and arsines.

The luminescent metal chelate is particularly preferably selected from metal chelates with bipyridyl or phenanthrolyl ligands. Examples of suitable metal chelates and the production thereof are described in EP-A-0 178 450, EP-A-0 255 534, EP-A-0 580 979 and WO 90/05301. Reference is herewith made to these disclosures. Ruthenium-(bipyridyl)₃ chelates are the most preferred metal chelates. These chelates are commercially available in the form of active ester derivatives for example from the Igen Inc. Company (Rockville, Md., USA).

When using a luminescent metal complex which is detectable by an electrochemiluminescence reaction as the marker group, the incorporation of at least one positive or/and negative charge carrier e.g. amino or carboxylate groups into the carrier chain or/and the spacer between metal complex and carrier chain has proven to be advantageous. The carrier chain particularly preferably contains one or several negative charges which can for example be generated by incorporation of glutamic acid or asparaginic acid during the synthesis. Also in the case of other marker or solid phase binding groups e.g. fluorescent groups or biotin it may be advantageous to incorporate charge carriers into the carrier chain or/and into the spacer.

A further example of preferred marker groups are fluorescent labels such as fluorescein, coumarin, rhodamine, resorufin, cyanine and derivatives thereof. When using fluorescent marker groups it has proven to be advantageous to use a helical structure of the carrier backbone which immobilizes the fluorescent marker groups with regard to spatial orientation and spacing in order to prevent fluorescence quenching by energy transfer. Examples of monomers which result in a suitable helical structure are proline or a peptidic nucleic acid derivative with a proline side group.

The conjugates according to the invention are produced by a process in which a polymeric carrier, preferably a peptide carrier, composed of monomeric units is synthesized on a solid phase in which (a) during the synthesis monomer derivatives are introduced at predetermined positions on the carrier which are covalently coupled to hapten molecules or/and marker or solid phase binding groups or/and (b) after the synthesis activated hapten molecules or/and marker or solid phase binding groups are coupled to reactive side groups of the carrier.

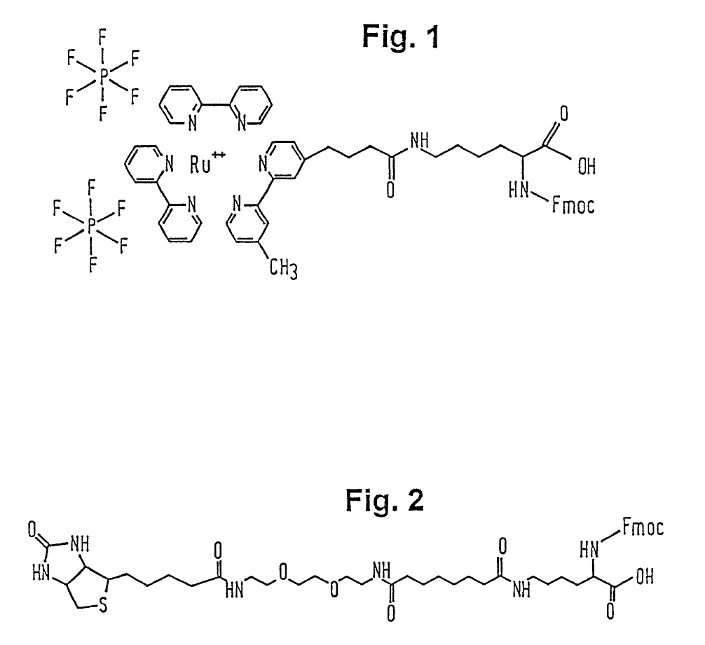

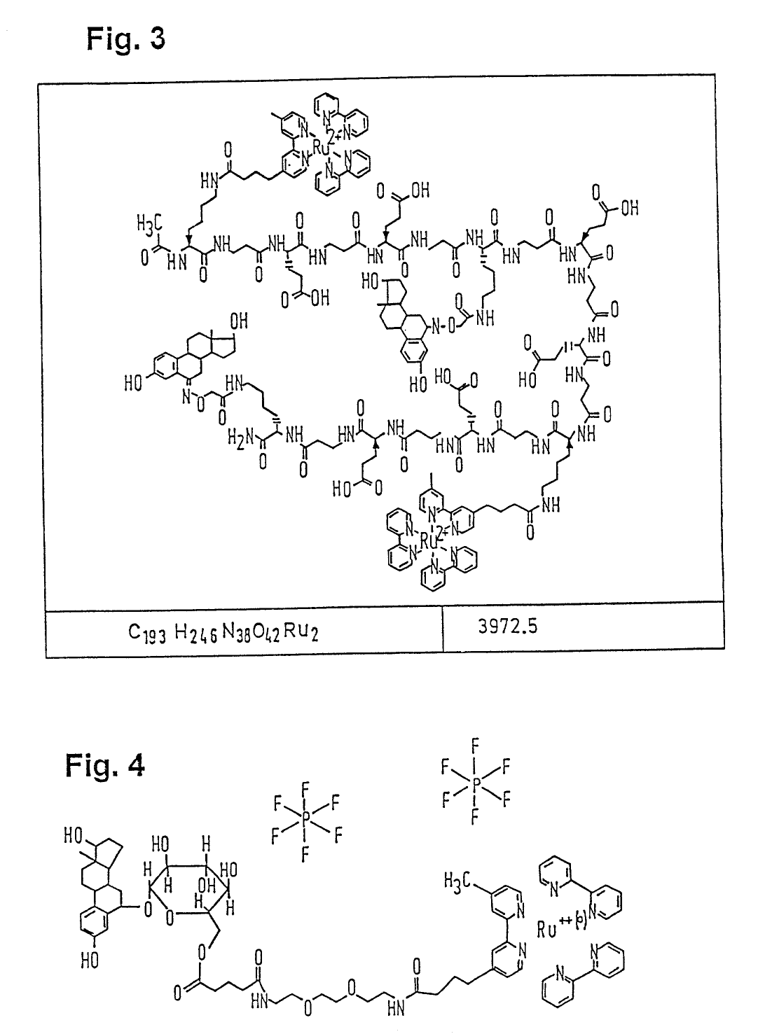

In variant (a) of the method according to the invention a monomer derivative is introduced during the solid phase synthesis which is covalently coupled to a hapten molecule or/and a marker or solid phase binding group preferably via a primary amino side group of a basic amino acid such as lysine or ornithine or via a thiol side group of an amino acid such as cysteine. The corresponding monomer derivatives are for example synthesized by coupling an activated hapten molecule or an activated marker or solid phase binding group e.g. an active ester derivative to a free primary amino group or a maleimide derivative to a free thiol group of optionally partially protected monomer derivatives e.g. amino acid derivatives. A preferred metal chelate-coupled lysine derivative is shown in FIG. 1. FIG. 2 shows a biotinylated lysine derivative.

The term “active ester” within the sense of the present invention encompasses activated ester groups which can react with free amino groups of peptides under such conditions that no interfering side reactions with other reactive groups of the peptide can occur. An N-hydroxysuccinimide ester is preferably used as the active ester derivative. Analogous p-nitrophenyl, pentafluorophenyl, imidazolyl or N-hydoxybenzotriazolyl esters can also be used in addition to N-hydroxysuccinimide esters.

The synthesis of derivatives of hapten, marker groups and solid phase binding groups that are suitable for incorporation into oligonucleotide carriers is described in Theisen et al. (Tetrahedron Letters 33 (1992), 5033-5036) using the fluorescent dye 5-carboxyfluorescein which is converted into a phosphoramidite derivative as an example. This phosphoramidite derivative can be incorporated at the 5′ end or/and the 3′ end of oligonucleotides or within the oligonucleotide sequence.

According to variant (b) of the process according to the invention the introduced group is coupled after cleaving protecting groups of the monomer derivatives used for the solid phase synthesis preferably to amino or/and thiol side groups especially preferably to primary amino side groups of the carrier.

The haptens and marker or solid phase binding groups are preferably introduced according to variant (a) i.e. by using monomer derivatives during the solid phase synthesis that are coupled to the group that is to be introduced in each case. According to this variant luminescent metal chelates, biotin or peptide haptens can for example be introduced without problems. However, in the case of sensitive fluorescent dyes or labels or other haptens such as steroids this procedure is unsuited since these substances can be destroyed under the conditions of the solid phase syntheses. In this case the conjugates are synthesized according to process variant (b) i.e. by subsequent coupling to the completed carrier molecule. Of course it is also possible to use a combination of process variants (a) and (b).

It is also possible to introduce two different groups according to variant (b) i.e. after completion of the synthesis e.g. a hapten and a marker group or a hapten and a solid phase binding group. In this connection the process according to variant (b) can for example be carried out in such a way that the first group to be introduced is coupled to amino side groups and the second group to be introduced is coupled to thiol side groups of the carrier molecule. On the other hand both groups to be introduced can each be coupled selectively to predetermined primary amino side groups of the carrier in which a monomer derivative with a first protecting group is used for the amino side group at positions of the carrier at which hapten molecules are to be coupled and a monomer derivative with a second protecting group for the amino side group is used at positions of the carrier at which marker or solid phase binding groups are to be coupled and the first and the second protecting group are selected in such a way that it enables a selective cleavage of the protecting groups and thus a selective coupling in two reaction steps. For this purpose the first and second protecting group can be selected from acid-labile amino protecting groups such as Boc or acid-stable protecting groups such as phenylacetyl.

In the process according to the invention the carrier molecule having the desired monomer sequence is synthesized on a solid phase. The peptide carriers are preferably produced using a commercial peptide synthesizer (e.g. the instruments A 431 or A 433 from Applied Biosystems). The synthesis is carried out according to known methods preferably starting at the carboxyl terminus of the peptide using amino acid derivatives. Amino acid derivatives are preferably used whose amino terminal group required for coupling is derivatized with a fluorenylmethyloxycarbonyl (Fmoc) residue. Reactive side groups of the amino acids used contain protecting groups that can be readily cleaved off after completion of the peptide synthesis. Preferred examples of this are protecting groups such as triphenylmethyl (Trt), t-butyl ether (tBu), t-butyl ester (OtBu), tert.-butoxycarbonyl (Boc), 2,2,5,7,8-penta-methylchroman-6-sulfonyl (Pmc) or phenylacetyl.

The amino side chains of lysine residues or of other amino acid derivatives with primary amino side groups that are located at positions of the peptide at which it is intended to introduce a hapten or label are covalently coupled to the group to be introduced according to variant (a).

In addition to the 20 natural amino acids the peptide can also contain artificial amino acids such as β-alanine, γ-amino-butyric acid, ε-amino-caproic acid, norleucine or ornithine. These artificial amino acids are used for the synthesis in a protected form analogously to the natural amino acids.

According to variant (b) of the process according to the invention the hapten or the label is introduced after completion of the synthesis by reacting the peptide after cleavage of protecting groups with the activated group desired in each case which reacts with free primary amino groups of the peptide. 1.5 to 4 equivalents of active ester are preferably used per free primary amino group. Subsequently the reaction product is purified, preferably by HPLC. The introduction of two different activated groups according to variant (b) is achieved by using two selectively cleavable protecting groups as elucidated above.

The peptide backbone of the conjugate has a non-immunologically reactive amino acid sequence i.e. an amino acid sequence which does not interfere with the test procedure in the intended application of the conjugate as an antigen in an immunological method of detection.

On the other hand the backbone of the carrier molecule can also be composed of nucleotides or peptidic nucleic acids. The synthesis of an oligonucleotide carrier molecule can be carried out in a commercial DNA synthesizer. The hapten molecules and the marker or solid phase binding groups are preferably introduced as phosphoramidite derivatives (Theisen et al., supra; Applied Biosystems, User Bulletin 67, FAM Amidite, May 1992) or/and they are subsequently coupled to free reactive side chains. Carrier molecules based on peptidic nucleic acids are synthesized analogously to a solid phase peptide synthesis e.g. according to the method described in WO92/20703. The hapten molecules or marker or solid phase binding groups can be introduced according to the methods described for peptide and oligonucleotide carriers.

The present invention also concerns the use of the conjugates as antigens in an immunological method if the hapten molecule is an immunologically reactive molecule or for DNA diagnostics if the hapten is a nucleic acid.

Conjugates which contain more than one hapten molecule can be used in immunological detection methods as polyhaptens.

A preferred embodiment of the invention concerns the use of the conjugates in an immunological method for the determination of specific antibodies in a sample liquid. Such antibodies are preferably determined which indicate an infection by microorganisms such as bacteria, viruses or protozoa. Antibodies directed against viruses e.g. antibodies directed against HIV or hepatitis viruses are particularly preferably determined. The sample liquid is preferably serum and particularly preferably human serum. In addition it is preferred that the conjugates according to the invention are used in an immunological method in a bridge test format.

Thus the present invention concerns a method for the immunological determination of a specific antibody in a sample liquid which is characterized in that (a) the sample liquid is incubated with at least one conjugate according to the invention which is directed against the antibody to be determined and (b) the antibody is detected via binding to the peptide.

The immunological method of determination according to the invention can in fact be carried out according to any known test format e.g. in a homogeneous immunoassay with a single reaction phase or in a heterogeneous immunoassay with more than one reaction phase. A heterogeneous test format is preferably used in which the presence of the antibody is detected in the presence of a solid phase. One embodiment of this test format is the so-called double antigen bridge test design. In this case the sample liquid is incubated in the presence of a reactive solid phase with two antigens directed against the antibody to be determined of which the first antigen carries a marker group and the second antigen is bound to the solid phase or is present in a form capable of binding to the solid phase. The first or/and the second antigen is a conjugate according to the invention. The antibody to be determined in the sample liquid is detected after optionally separating the solid phase from the incubation liquid by determining the label in the solid phase or/and in the liquid phase. The first antigen is preferably a conjugate labelled with a luminescent metal chelate or a fluorescent group. The second antigen is preferably labelled with biotin and is capable of binding to a solid phase which is coated with streptavidin or avidin.

The test procedure preferably comprises mixing the sample liquid with the first labelled antigen and the second antigen on the solid phase in order to obtain a labelled immobilized complex of first antigen, antibody and solid phase-bound second antigen. Compared to other test formats for detecting antibodies, the bridge test format leads to an improvement in sensitivity i.e. all immunoglobulin classes such as IgG, IgM, IgA and IgE are detected as well as in specificity i.e. the unspecific reactivity is reduced.

A second preferred embodiment of the invention concerns the use of the conjugates in a competitive immunoassay. Competitive immunoassays are generally used to detect lower molecular analytes and can in principle be carried out in two test formats the “labelled antibody” format and the “labelled analogue” format. In the “labelled antibody” format the sample liquid containing the analyte to be determined is incubated with a hapten capable of binding to a solid phase which competes immunologically with the analyte and a labelled receptor, e.g. an antibody, directed against the hapten and the analyte. In this test format the label bound to the solid phase is inversely proportional to the concentration of the analyte. In the “labelled analogue” format the sample liquid containing the analyte to be determined is incubated with a labelled hapten that immunologically competes with the analyte and a receptor directed against the analyte and the hapten which is capable of binding to a solid phase. The amount of solid phase-bound labelled hapten is then inversely proportional to the concentration of free analyte to be determined.

Thus the present invention concerns a method for the detection of an analyte in a sample liquid based on the principle of a competitive immunoassay in a “labelled analogue” format which is characterized in that (a) the sample liquid is incubated in the presence of a reactive solid phase with a conjugate according to the invention which contains a marker group and with a receptor which is bound to the solid phase or is capable of binding to the solid phase and which can enter into a specific immunological reaction with the analyte and the hapten component of the conjugate, (b) the solid phase is optionally separated from the incubation liquid and (c) the presence or/and the amount of analyte in the sample liquid is determined by measuring the marker component of the conjugate in the solid phase or/and in the incubation liquid. A biotinylated antibody or a biotinylated antibody fragment is preferably used as the immobilizable receptor and a solid phase coated with streptavidin or avidin is preferably used as the reactive solid phase.

Yet a further subject matter of the present invention is a method for detecting an analyte in a sample liquid based on the principle of a competitive immunoassay in a “labelled antibody” format which is characterized in that (a) the sample liquid is incubated in the presence of a reactive solid phase with a conjugate according to the invention which contains a solid phase binding group and with a receptor which carries a marker group and which can enter into a specific immunological reaction with the analyte and the hapten component of the conjugate, (b) the solid phase is optionally separated from the incubation liquid and (c) the presence or/and the amount of analyte in the sample liquid is determined by measuring the marker component of the receptor in the solid phase or/and in the incubation liquid. A biotinylated conjugate and a solid phase coated with streptavidin or avidin is preferably used. An antibody or an antibody fragment is preferably used as the labelled receptor.

The luminescent metal chelate groups are preferably detected by means of electrochemiluminescence in which luminescent species are generated electrochemically on the surface of an electrode. The luminescence can be detected qualitatively or/and quantitatively. Examples for carrying out luminescence assays can be found in EP-A-0 580 979, WO 90/05301, WO 90/11511 and WO 92/14138. Reference is herewith made to the processes and devices for luminescence assays disclosed therein. The solid phase in electrochemiluminescence assays is preferably composed of microparticles and particularly preferably of magnetic microparticles that are provided with a coating that interacts with the second antigen on the solid phase. The microparticles are preferably coated with streptavidin.

Electrochemiluminescence is preferably measured in the presence of a reducing agent for the metal complex e.g. an amine. Aliphatic amines are preferred, in particular primary, secondary and tertiary alkylamines whose alkyl groups each have one to three carbon atoms. Tripropylamine is particularly preferred. However, the amine can also be an aromatic amine such as aniline or a heterocyclic amine.

Furthermore a non-ionic surface active agent e.g. an ethoxylated phenol can optionally be present as an amplifier. Such substances are for example commercially available under the names Triton X100 or Triton N-401.

On the other hand the luminescent metal chelate group can also be detected by fluorescence in which the metal chelate is excited by irradiation with light of a suitable wavelength and the fluorescence radiation resulting therefrom is measured. Examples for carrying out fluorescence assays are given in EP-A-0 178 450 and EP-A-0 255 534. Reference is herewith made to this disclosure.

Detection of fluorescent groups which like the luminescent metal chelates are also preferred marker groups of the conjugates according to the invention can—as stated above—be carried out by excitation and measurement of the fluorescence in a known manner.

Yet a further subject of the present invention is an immunological reagent which contains at least one labelled or solid phase-bindable conjugate according to the invention. A reagent for the immunological determination of a specific antibody based on the principle of a double antigen bridge test contains (a) a labelled conjugate according to the invention or/and (b) a further conjugate according to the invention which is bound to a solid phase or is present in a form capable of binding to a solid phase.

A reagent for determining an analyte, preferably a lower molecular analyte based, on the principle of a competitive immunoassay contains a labelled conjugate (“labelled analogue” format) or a solid phase bindable conjugate (“labelled antibody” format) which competes immunologically with the analyte to be determined for binding to a receptor. The reagent for a competitive immunoassay preferably contains spatially separated from the conjugate according to the invention either a labelled receptor (“labelled antibody” format) or a solid phase bindable receptor (“labelled analogue” format) which can react immunologically with the analyte to be determined and with the conjugate according to the invention.

The invention is further elucidated by the following examples and figures. They show:

FIG. 1 a metal chelate-lysine derivative

FIG. 2 a biotin-lysine derivative

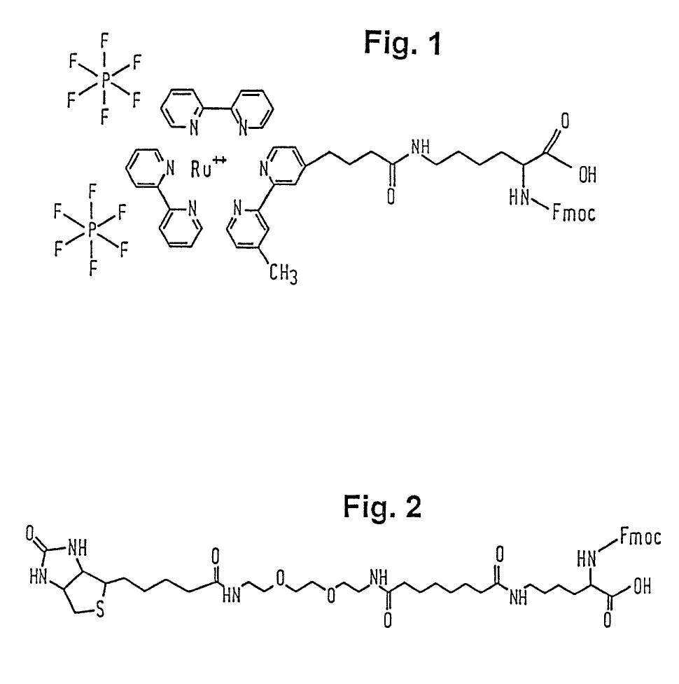

FIG. 3 a conjugate according to the invention

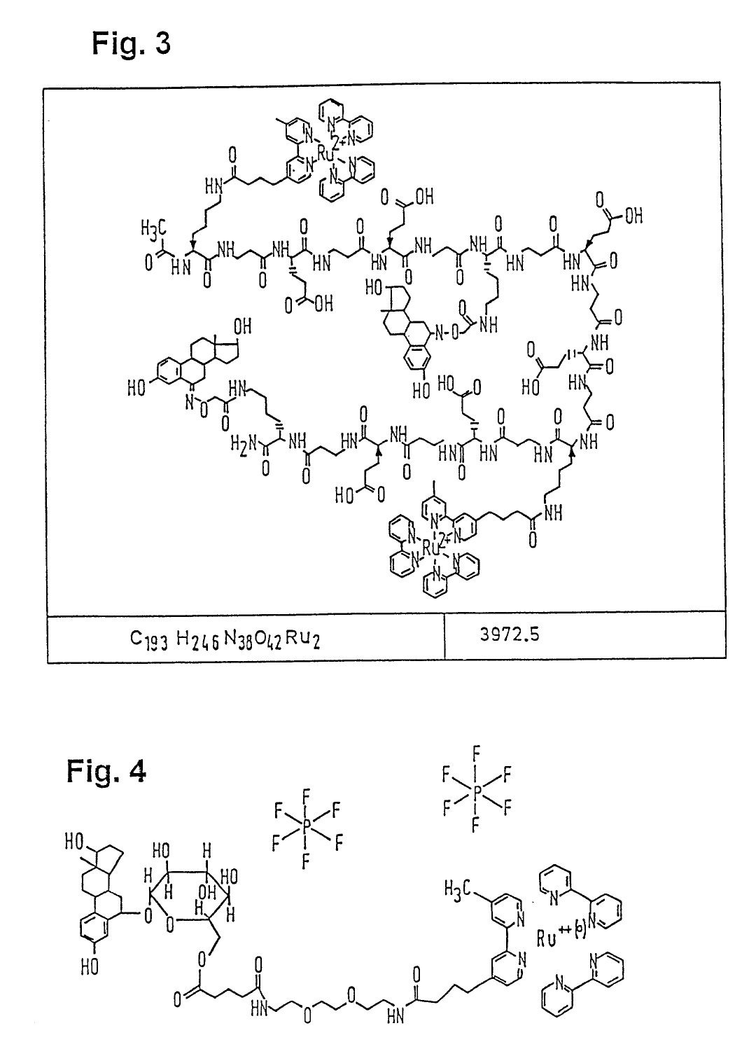

FIG. 4 a reference conjugate

### EXAMPLE 1

Production of a Metal Chelate-Lysine Derivative

6 mmol of the ruthenium complex Ru(bipyridine)₂ (bipyridine-CO-N-hydroxysuccinimide ester) according to EP-A-0 580 979 was dissolved in 50 ml dimethylformamide and a solution of α-Fmoc lysine was added dropwise. After removing the solvent, the residue was dissolved in a small amount of acetone, admixed with 300 ml chloroform and briefly heated to boiling. After separating the solvent, the compound shown in FIG. 1 was obtained as a solid.

### EXAMPLE 2

Production of Metal Chelate-Labelled and Biotinylated Peptides

The metal chelate-labelled and biotinylated peptides were produced by means of fluorenylmethyloxycarbonyl-(Fmoc) solid phase peptide synthesis on a batch peptide synthesizer e.g. from Applied Biosystems A431 or A433. For this 4.0 equivalents of the amino acid derivatives shown in Table 1 were used in each case:

TABLE 1

| | |
|---|---|
| A | Fmoc-Ala-OH |
| C | Fmoc-Cys(Trt)-OH |
| D | Fmoc-Asp(OtBu)-OH |
| E | Fmoc-Glu(OtBu)-OH |
| F | Fmoc-Phe-OH |
| G | Fmoc-Gly-OH |
| H | Fmoc-His(Trt)-OH |
| I | Fmoc-Ile-OH |
| K | Fmoc-Lys(Boc)-OH |
| L | Fmoc-Leu-OH |
| M | Fmoc-Met-OH |
| N | Fmoc-Asn(Trt)-OH |
| P | Fmoc-Pro-OH |
| Q | Fmoc-Gln(Trt)-OH |
| R | Fmoc-Arg(Pmc)-OH |
| S | Fmoc-Ser(tBu)-OH |
| T | Fmoc-Thr(tBu)-OH |
| U | Fmoc-βAlanine-OH |
| V | Fmoc-Val-OH |
| W | Fmoc-Trp-OH |
| Y | Fmoc-Tyr(tBu)-OH |
| Z | Fmoc-ε-aminocaproic acid-OH |
| Nle | Fmoc-ε-norleucine-OH |
| Abu | Fmoc-γ-aminobutyric acid-OH |

Introduction of metal chelate and biotin groups into the peptide sequence was carried out by direct incorporation of metal chelate-coupled or biotin-coupled amino acid derivatives e.g. within the sequence via a lysine residue ε-derivatized with a metal chelate active ester (FIG. 1) or via a biotin-derivatized lysine residue (FIG. 2) or N-terminally by using a corresponding α-derivatized amino acid residue.

The amino acids or amino acid derivatives were dissolved in N-methylpyrrolidone. The peptide was synthesized on 400-500 mg 4-(2′,4′-dimethoxyphenyl-Fmoc-aminomethyl)phenoxy resin (Tetrahedron Letters 28 (1987), 2107) with a loading of 0.4-0.7 mmol/g (JACS 95 (1973), 1328). The coupling reactions were carried out for 20 minutes in dimethylformamide as a reaction medium with 4 equivalents dicyclohexylcarbodiimide and 4 equivalents of N-hydroxybenzotriazole relative to the Fmoc-amino acid derivative. The Fmoc group was cleaved in 20 minutes after each synthesis step using 20% piperidine in dimethylformamide.

The release of the peptide from the support and the cleavage of the acid-labile protecting groups was achieved in 40 min at room temperature with 20 ml trifluoro acetic acid, 0.5 ml ethanedithiol, 1 ml thioanisole, 1.5 g phenol and 1 ml water. The reaction solution was subsequently admixed with 300 ml cooled diisopropyl ether and kept at 0° C. for 40 min to completely precipitate the peptide. The precipitate was filtered, washed again with diisopropyl ether, dissolved in a small amount of 50% acetic acid and lyophilized. The crude material obtained was purified in ca. 120 min. by means of preparative HPLC on delta-PAK RP C18 material (column 50×300 mm, 100 Å, 15μ) using an appropriate gradient (eluant A: water, 0.1% trifluoroacetic acid, eluant B: acetonitrile, 0.1% trifluoroacetic acid). The identity of the eluted material was checked by means of ion spray mass spectrometry.

Acid-stable phenylacetyl protecting groups were removed at room temperature enzymatically using immobilized or soluble penicillin G-amidase in aqueous solution with an organic solvent component.

### EXAMPLE 3

Coupling to Haptens

The metal chelate-labelled or biotinylated peptide was dissolved in dimethylformamide and a slight excess (ca. 30%) of the activated hapten (e.g. N-hydroxysuccinimide ester) was added with regard to the positions on the peptide capable of coupling (e.g. amino side groups of lysine). It was stirred for 1 hour at room temperature, the solvent was removed in a high vacuum and the peptide was purified by means of preparative HPLC.

Conjugates having the following structure were prepared using the metal chelate-lysine derivative shown in FIG. 1 as the marker group and estradiol (E2) as the hapten:

I: AcK(BPRu)UEUEUK(E2)UEUEUK(BPRu)UEUK(E2)-NH₂

II: AcK(BPRu)UEUEUK(E2)UEUEUK(BPRu)U-NH₂

The conjugate I is shown in FIG. 3. The amino terminus of the peptide chains is protected by acetyl (Ac). The carboxyl terminus is present as an acid amide group.

The metal chelate and hapten molecules are in each case coupled to the peptide chain via the ε-amino side group of the lysines.

A conjugate having the sequence of conjugate I is synthesized in an analogous manner using testosterone as a hapten molecule.

The structure of the conjugates produced was examined and confirmed by means of ¹H-NMR (500 MHz).

### EXAMPLE 4

Determination of Estradiol in Serum

A competitive two-step assay based on the “labelled analogue” format was carried out to determine estradiol in human serum. For this 90 μl solution 1 (0.69 nmol/l conjugate I according to the invention (FIG. 3) or 1.68 nmol/l reference conjugate (FIG. 4) each in 50 mmol/l 4-morpholine ethane sulfonic acid (MES), pH 6.8, 0.1% bovine serum albumin, 0.1% Thesit, 0.01% methylisothiazolone, 0.1% oxypyrion, 30 ng/ml detachment reagent dihydrotestosterone) was incubated for 10 min at 37° C. in a polystyrene vessel together with 50 μl sample (serum sample or estradiol standard). Subsequently 90 μl solution 2 (0.7 μg/ml or 1.4 μg/ml conjugate of biotin and polyclonal anti-estradiol rabbit Fab′ in 50 mmol/l MES buffer, pH 6.0) and 50 μl bead suspension (720 μg/ml streptavidin-coated magnetic particles, Dynal Company) were added in succession.

After incubating for a further 10 minutes at 37° C., 150 μl of the mixture was transferred into a measuring cell. There the bead particles and the ruthenium label adhering thereto was magnetically concentrated on the electrode surface and the chemiluminescence signal generated electrochemically was detected at 28° C.

The result of this experiment shown in Table 2 shows that the conjugate according to the invention has a considerably improved test performance with regard to sensitivity and to lower detection limit compared to the reference conjugate.

TABLE 2

| | Conjugate I | Conjugate |
|---|---|---|
| Antigen | (invention) | (reference) |
| Antigen concentration (nmol/l) | 0.69 | 1.68 |
| Ruthenium complex concentration | 1.68 | 1.68 |
| (nmol/l) | | |
| Concentration antiserum | 0.7 | 1.4 |
| (μg/ml) | | |
| Counts Standard F | 80412 | 87709 |
| Counts Standard B | 653420 | 1223219 |
| Counts Standard A | 861140 | 1445270 |
| Ratio B/A | 0.759 | 0.847 |
| Ratio F/A | 0.093 | 0.061 |
| Ratio F/E | 0.546 | 0.442 |
| lower detection limit | 15.9 | 25.1 |
| pg/ml (3% CV) | | |

What is claimed is:

1. A process for producing a conjugate comprising (a) forming a carrier on a solid phase by linking together monomeric units, and (b) introducing into the carrier at predetermined positions 1-10 additional monomeric units covalently bound to hapten molecules and 1-10 additional monomeric units covalently bound to marker groups or solid phase binding groups, whereby the conjugate comprises a maximum of 100 monomeric units selected from the group consisting of nucleotides, nucleotide analogues and amino acids.

2. A process for producing a conjugate comprising (a) forming a carrier on a solid phase by linking together monomeric units, (b) introducing into the carrier at predetermined positions additional monomeric units comprising reactive side groups and protecting groups for said side groups, (c) cleaving said protecting groups, and (d) coupling 1-10 hapten molecules and 1-10 marker groups or solid phase binding groups to said reactive side groups, whereby the conjugate comprises a maximum of 100 monomeric units selected from the group consisting of nucleotides, nucleotide analogues and amino acids.

3. The process as claimed in claim 1, wherein the monomeric units are amino acids.

4. The process as claimed in claim 2, wherein the monomeric units are amino acids and 2-10 hapten molecules are coupled in step (d).

5. The process as claimed in claim 1, wherein the monomeric units covalently bound to hapten molecules and the monomeric units covalently bound to marker groups or solid phase binding groups are bound via primary amino groups or thiol groups.

6. The process as claimed in claim 2, wherein the reactive side groups are primary amino groups the protective groups are selectively cleavable.

7. The process as claimed in claim 6, wherein the protective groups are selected from the group consisting of acid-labile groups and acid-stable groups.

8. A process for producing a conjugate comprising
(a) forming a carrier on a solid phase by linking together monomeric units,
(b) introducing into the carrier at predetermined positions 1-10 additional monomeric units covalently bound to hapten molecules and 1-10 additional monomeric units covalently bound to marker groups or solid phase binding groups, and
(c) introducing into the carrier at predetermined positions additional monomeric units comprising reactive side groups and protecting groups for said side groups, cleaving said protecting groups, and coupling 1-10 hapten molecules and 1-10 marker groups or solid phase binding groups to said reactive side groups,
whereby the conjugate comprises a maximum of 100 monomeric units selected from the group consisting of nucleotides, nucleotide analogues and amino acids.
